(19) United States
(12) Patent Application Publication
(10) Pub. No.: US 2026/0027342 A1
(43) Pub. Date: Jan. 29, 2026

(54) VENT PLUGS AND RELATED CATHETER SYSTEMS

(71) Applicant: Becton, Dickinson and Company, Franklin Lakes, NJ (US)

(72) Inventors: Edwin Jeyaseelan A., Tamil Nadu (IN); Prasad Govindaraj, Tamil Nadu (IN); Meenakshi Sankar Gomathinayagam, Tamil Nadu (IN); Sankaranarayanan Thirumoorthy, Tamil Nadu (IN); S. Ray Isaacson, Layton, UT (US)

(21) Appl. No.: 18/784,507

(22) Filed: Jul. 25, 2024

(51) Int. Cl. A61M 39/20; A61M 25/06; A61M 39/10

(52) U.S. Cl. CPC A61M 39/20; A61M 25/0606; A61M 39/10

(57) ABSTRACT

A catheter system may include a catheter adapter, which may include a distal end and a proximal end. The catheter system may include a catheter and a vent plug in fluid communication with the catheter. The vent plug may include a distal end having a hole. The hole may be sized to pass air but not blood. The vent plug may include a proximal end and a lumen extending proximally from the hole through the proximal end of the vent plug. The vent plug may include an air-permeable plug disposed within the lumen. The air-permeable plug may include cellulose or polyurethane foam. The distal end of the vent plug may include a protrusion forming a stepped surface. The air-permeable plug may extend along a portion of the lumen or may extend along an entire length of the lumen from the hole to the proximal end.

### BACKGROUND

A common type of catheter assembly includes a catheter that is over-the-needle. As its name implies, the catheter that is over-the-needle may be mounted over an introducer needle having a sharp distal tip. The catheter assembly may include a catheter adapter and the catheter extending distally from the catheter adapter. The catheter and the introducer needle may be assembled such that the distal tip of the introducer needle extends beyond the distal tip of the catheter with a bevel of the introducer needle facing up away from skin of the patient immediately prior to insertion into the skin. The catheter and the introducer needle are generally inserted at a shallow angle through the skin into a blood vessel of the patient.

In order to verify proper placement of the introducer needle and/or the catheter in the blood vessel, a clinician may confirm that there is flashback of blood in a flashback chamber of the catheter assembly and/or in a space between the introducer needle and the catheter. The flashback of blood travels into the flashback chamber or the space between the introducer needle and the catheter because a vent plug permits air to escape the catheter assembly as blood enters the catheter assembly.

Once placement of the introducer needle within the blood vessel has been confirmed by visualizing the flashback, the clinician may remove the introducer needle, leaving the catheter in place in the blood vessel for future blood withdrawal or fluid infusion. The clinician may also clamp the catheter assembly to halt progression of blood through the catheter assembly, remove the vent plug, and replace the vent plug with another vascular access device such as an infusion set connection or a Luer access port. The clinician may then unclamp the catheter assembly, flush the blood from the catheter assembly back into the vasculature of the patient, and reclamp the catheter assembly.

The subject matter claimed the present disclosure is not limited to embodiments that solve any disadvantages or that operate only in environments such as those described above. Rather, this background is only provided to illustrate one example technology area where some implementations described the present disclosure may be practiced.

### SUMMARY

The present disclosure relates generally to vent plugs and related catheter systems. According to a first set of embodiments, a vent plug for a catheter system may include a distal end, which may include no more than one hole. In some embodiments, the hole may be sized to pass air but not blood. In some embodiments, the vent plug may include a proximal end. In some embodiments, the vent plug may include a lumen extending proximally from the hole through the proximal end. In some embodiments, the vent plug may include an air-permeable plug disposed within a proximal end of the lumen. In some embodiments, the air-permeable plug may include cellulose.

In some embodiments, a diameter of the hole may be less than a diameter of the lumen. In some embodiments, the distal end of the vent plug may include a protrusion forming a stepped surface. In some embodiments, the hole may extend through the protrusion. In some embodiments, the hole may include a cylindrical bore hole. In some embodiments, the lumen may be cylindrical or generally cylindrical.

According to a second set of embodiments, a vent plug for a catheter system may include a distal end having a hole. In some embodiments, the hole may be sized to pass air but not blood. In some embodiments, the vent plug may include a proximal end. In some embodiments, the vent plug may include a lumen extending proximally from the hole through the proximal end. In some embodiments, the vent plug may include an air-permeable plug disposed within the lumen. In some embodiments, the air-permeable plug may include polyurethane foam.

In some embodiments, the air-permeable plug may extend along a portion of the lumen and may be spaced apart from the hole. In some embodiments, the air-permeable plug may extend along an entire length of the lumen from the hole to the proximal end. In some embodiments, a diameter of the hole may be less than a diameter of the lumen. In some embodiments, the hole may include a cylindrical bore hole. In some embodiments, the lumen may be cylindrical or generally cylindrical.

According to a third set of embodiments, a catheter system may include a catheter adapter, which may include a distal end and a proximal end. In some embodiments, the catheter system may include a catheter extending distally from the distal end of the catheter adapter. In some embodiments, the catheter system may include a vent plug in fluid communication with the catheter.

In some embodiments, the vent plug may include a distal end having a hole that may be sized to pass air but not blood. In some embodiments, the vent plug may include a proximal end. In some embodiments, the vent plug may include an air-permeable plug disposed within a proximal end of the lumen. In some embodiments, the air-permeable plug may include cellulose or polyurethane foam.

In some embodiments, the catheter system may include an extension tube and a connector at a proximal end of the extension tube. In some embodiments, the vent plug may be coupled to the connector. In some embodiments, the catheter adapter may include a side port disposed between the distal end of the catheter adapter and the proximal end of the catheter adapter. In some embodiments, a distal end of the extension tube may be coupled to the side port, and the extension tube may extend from the side port.

In some embodiments, a distal end of the extension tube may be coupled to the proximal end of the catheter adapter, and the extension tube may extend from the proximal end of the catheter adapter. In some embodiments, the catheter system may include a needle assembly coupled to the proximal end of the catheter adapter. In some embodiments, the vent plug may be coupled to a proximal end of the needle assembly.

In some embodiments, the hole may include a cylindrical bore hole. In some embodiments, a diameter of the hole may be less than a diameter of the lumen. In some embodiments, the distal end of the vent plug may include a protrusion forming a stepped surface. In some embodiments, the hole may extend through the protrusion. In some embodiments, the air-permeable plug may extend along a portion of the lumen and may be spaced apart from the hole. In some embodiments, the air-permeable plug may extend along an entire length of the lumen from the hole to the proximal end.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present disclosure. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1A is an upper perspective view of an example catheter system, according to some embodiments;

FIG. 1B is an upper perspective view of an example connector, according to some embodiments;

FIG. 1C is an upper perspective view of another catheter system, according to some embodiments;

FIG. 1D is a side view of another catheter system, according to some embodiments;

FIG. 1E is a cross-sectional view of the other catheter system of FIG. 1D, according to some embodiments;

FIG. 2A is a lower perspective view of an example vent plug, according to some embodiments;

FIG. 2B is a cross-sectional view of the vent plug, according to some embodiments;

FIG. 2C is an upper perspective view of the vent plug, according to some embodiments;

FIG. 3A is a lower perspective view of the vent plug having a protrusion at a distal end, according to some embodiments;

FIG. 3B is a cross-sectional view of the vent plug illustrating the protrusion at the distal end, according to some embodiments;

FIG. 4A is a lower perspective view of another vent plug, according to some embodiments;

FIG. 4B is a cross-sectional view of the other vent plug, according to some embodiments;

FIG. 5A is a lower perspective view of the other vent plug, illustrating an example air-permeable plug extending through an entirety of a lumen, according to some embodiments; and

FIG. 5B is a cross-sectional view of the other vent plug, illustrating the air-permeable plug extending through the entirety of the lumen, according to some embodiments.

### DESCRIPTION OF EMBODIMENTS

Regarding terms used in the present disclosure, it should be understood the terms are for the purpose of describing some particular embodiments, and the terms do not limit the scope of the concepts provided in the present disclosure. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Singular forms of “a,” “an,” and “the” include plural references unless the context clearly dictates otherwise. Also, the words “including,” “has,” and “having,” as used in the present disclosure, including the claims, shall have the same meaning as the word “comprising.”

As used in the present disclosure, the term “proximal” refers to a location with respect to a device during normal use that is closest to the clinician and farthest from the patient. With respect to “proximal,” a “proximal portion” or a “proximal end portion” of, for example, a catheter disclosed in the present disclosure includes a portion of the catheter intended to be near a clinician when the catheter is used on a patient. A “proximal end” of, for example, the catheter includes an end of the catheter intended to be near the clinician when the catheter is used on the patient.

The term “distal” refers to a location with respect to a device during normal use that is farthest from the clinician and closest to the patient. With respect to “distal,” a “distal portion” or a “distal end portion” of, for example, a catheter disclosed in the present disclosure includes a portion of the catheter intended to be near or in a patient when the catheter is used on the patient. A “distal end” of, for example, the catheter includes an end of the catheter intended to be near or in the patient when the catheter is used on the patient.

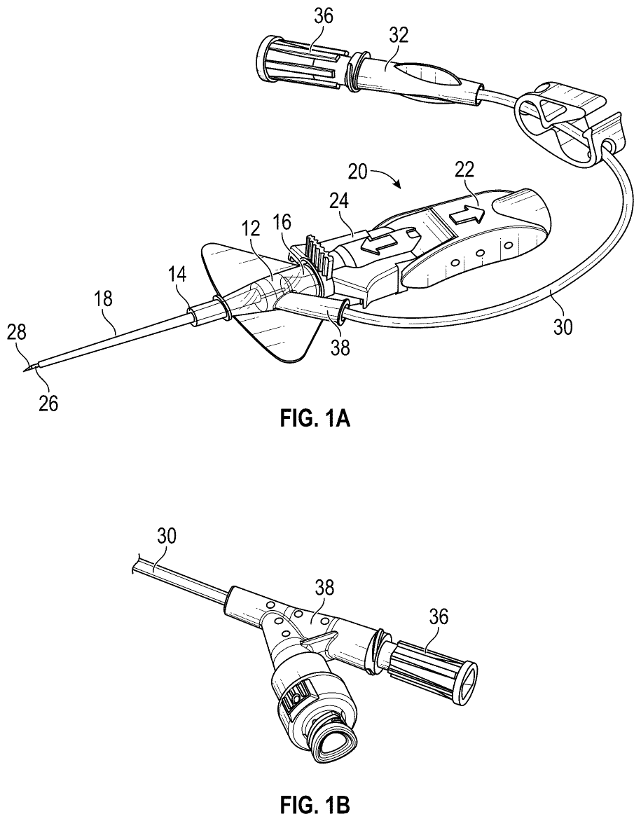

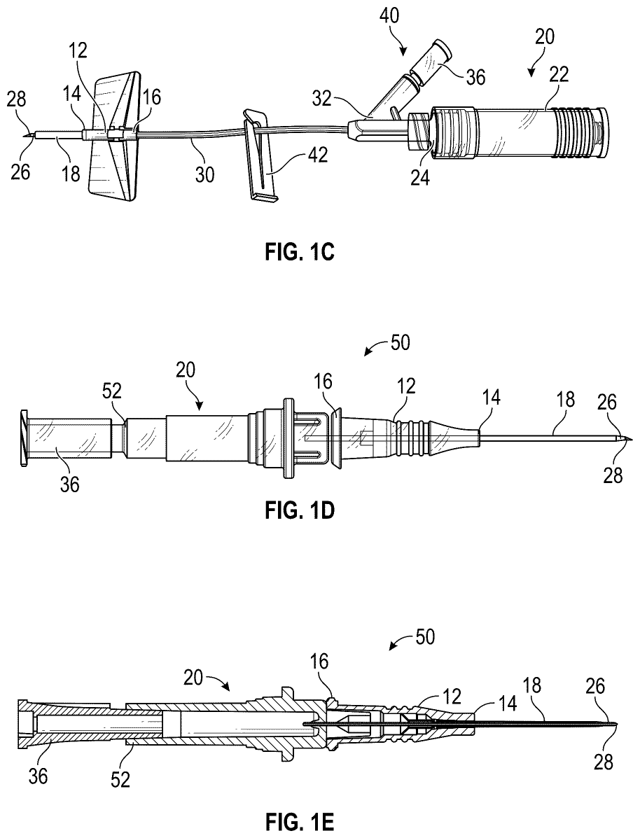

In some embodiments, a vent plug may be disposed at various locations within a particular catheter system in order to provide venting to the catheter system and facilitate blood flashback in response to insertion of a catheter within a blood vessel. Referring now to FIGS. 1A-1B, a catheter system 10 may include a catheter adapter 12, which may include a distal end 14 and a proximal end 16. In some embodiments, the catheter system 10 may include a catheter 18 extending distally from the distal end 14 of the catheter adapter 12.

In some embodiments, the catheter system 10 may include a needle assembly 20 coupled to the proximal end 16 of the catheter adapter 12. In some embodiments, the needle assembly 20 may include a needle hub 22 and a needle safety housing 24, which may include a needle safety clip. In some embodiments, a proximal end of an introducer needle 26 may be fixed within the needle hub 22, and the introducer needle 26 may extend through the catheter 18 in an insertion configuration such that a sharp distal tip 28 of the introducer needle 26 is distal to the catheter 18. In some embodiments, after the catheter 18 is positioned within a blood vessel using the introducer needle 26, the needle hub 22 may be retracted proximally such that the sharp distal tip 28 becomes trapped in the needle safety housing 24 and the needle assembly 20 may be removed from the catheter system 10.

In some embodiments, the catheter system 10 may include an extension tube 30 and a connector 32 at a proximal end of the extension tube 30. In some embodiments, the catheter system 10 may include a vent plug 36 in fluid communication with the catheter 18. In some embodiments, the vent plug 36 may be coupled to the connector 32. In some embodiments, a body of the vent plug 36 may be constructed or polypropylene or another suitable material. In some embodiments, the catheter adapter 12 may include a side port 38 disposed between the distal end 14 of the catheter adapter 12 and the proximal end 16 of the catheter adapter 12. In some embodiments, a distal end of the extension tube 30 may be coupled to the side port 38, and the extension tube 30 may extend from the side port 38.

In some embodiments, in response to the catheter 18 being inserted into the blood vessel, flashback of blood may flow into the introducer needle 26, through a notch in the introducer needle 26, into the catheter adapter 12, through the side port 38, into the extension tube 30, and to or towards the connector 32. In some embodiments, the vent plug 36 may provide air venting such that the flashback of blood is possible. In some embodiments, a clamp (not illustrated) may be placed on the extension tube 30.

In some embodiments, the connector 32 may include a single port, as illustrated, for example, in FIG. 1A. In other embodiments, the connector 32 may include multiple ports, as illustrated, for example, in FIG. 1B. In some embodiments, the catheter system 10 may include one or more features similar to the BD NEXIVA™ Closed IV Catheter System or the BD NEXIVA™ DIFFUSICS™ Closed IV Catheter System, both available from Becton, Dickinson & Company of Franklin Lakes, New Jersey.

Referring now to FIG. 1C, a catheter system 40 is illustrated, according to some embodiments. In some embodiments, the catheter system 40 may be similar or identical to the catheter system 10 of FIGS. 1A-1B in terms of one or more features and/or operation. In some embodiments, a distal end of an extension tube 30 may be coupled to a proximal end 16 of a catheter adapter 12, and the extension tube 30 may extend from the proximal end of the catheter adapter 12.

In some embodiments, in response to a catheter 18 being inserted into the blood vessel, flashback of blood may flow into an introducer needle 26, through a notch in the introducer needle 26, into the catheter adapter 12, through the proximal end 16, into the extension tube 30, and to or towards a connector 32. In some embodiments, the connector 32 may include a single port or multiple ports. In some embodiments, a vent plug 36 may provide air venting such that the flashback of blood is possible. In some embodiments, a clamp 42 may be placed on the extension tube 30.

In some embodiments, a needle safety housing 24 may be disposed within a needle hub 22. In some embodiments, the needle safety housing 24 may be telescoping and configured to expand when the needle hub 22 is retracted proximally such that a sharp distal tip 28 of the introducer needle 26 becomes trapped in the needle safety housing 24. In some embodiments, the catheter system 40 may include one or more features similar to the BD SAF-T-INTIMA™ Closed IV Catheter System, available from Becton, Dickinson & Company of Franklin Lakes, New Jersey.

Referring now to FIG. 1D, in some embodiments, a catheter system 50 is illustrated, according to some embodiments. In some embodiments, the catheter system 50 may be similar or identical to the catheter system 10 of FIGS. 1A-1B and/or the catheter system 40 of FIG. 1C in terms of one or more features and/or operation. In some embodiments, the vent plug 36 may be coupled to a proximal end 52 of a needle assembly 20, as illustrated, for example, in FIGS. 1D-1E. In some embodiments, the catheter system 50 may include one or more features similar to the BD INSYTE™ AUTOGUARD™ Shielded IV Catheter, available from Becton, Dickinson & Company of Franklin Lakes, New Jersey.

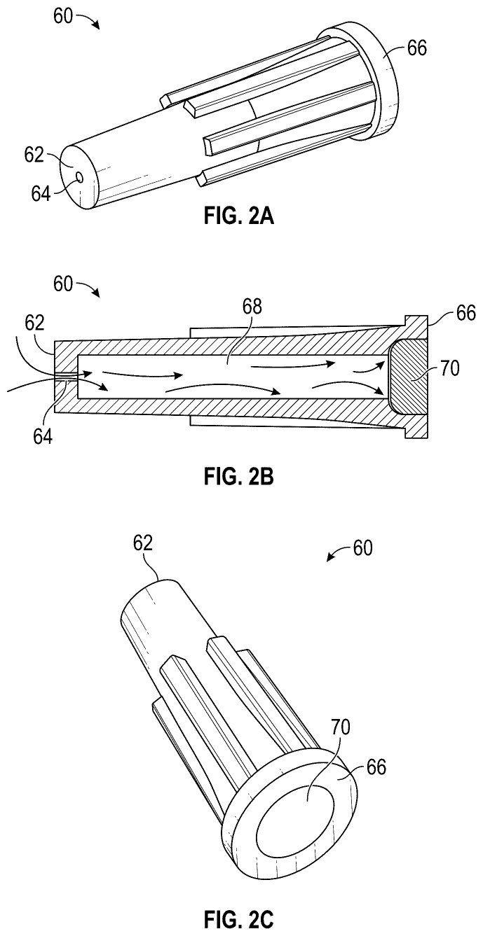

Referring now to FIGS. 2A-2C, a vent plug 60 is illustrated, according to some embodiments. In some embodiments, the vent plug 60 may correspond to the vent plug 36 of FIGS. 1A-1D. In some embodiments, the vent plug 60 may include a distal end 62, which may include no more than one hole 64. In some embodiments, a single hole as opposed to multiple holes may provide the clinician with added time to put on a cap on the vent plug 60 for closure before a risk of blood leaking from the vent plug 60 through an air-permeable plug. In some embodiments, it takes more than 15 seconds after blood enters the vent plug 60 for any blood to leak through the air-permeable plug. In some embodiments, blood may leak through the hole 64 into the lumen 68 and/or fill the lumen 68. However, the single hole and/or a small diameter of the single hole may increase venting time and a time it takes to fill the lumen 68 and/or blood to leak through the air-permeable plug. In some embodiments, the single hole and/or the small diameter of the single hole may provide the clinician more time to perform next steps such as capping the vent plug 60. In some embodiments, the hole 64 may include a bore hole, which may be cylindrical. In some embodiments, the hole 64 may be sized to pass air but not blood such that blood does not leak into the vent plug 60.

In some embodiments, the vent plug 60 may include a proximal end 66. In some embodiments, the vent plug 60 may include a lumen 68 extending proximally from the hole 64 through the proximal end 66. In some embodiments, the vent plug 60 may include an air-permeable plug 70 disposed within a proximal end of the lumen 68. In some embodiments, the air-permeable plug 70 may include cellulose to provide filtration of air but not blood. In some embodiments, the cellulose may have better absorption than filter paper and thus a time it takes for blood to travel through the vent plug 60 and leak from the air-permeable plug 70 may be increased. In some embodiments, the air-permeable plug 70 may be secured within the proximal end 66 of the vent plug 60 in an interference fit.

In some embodiments, a diameter of the hole 64 may be less than a diameter of the lumen 68, which may increase venting time and in turn provide the clinician more time to perform next steps such as capping the vent plug 60. In some embodiments, the diameter of the hole 64 may be less than an inner lumen of the introducer needle, which may increase the venting time and in turn provide the clinician more time to perform next steps such as capping the vent plug 60. In some embodiments, the diameter of the hole 64 may be between 0.5 millimeters and 0.8 millimeters, inclusive, which may facilitate the increase in the venting time. In some embodiments, the diameter of the hole 64 may be less than 0.5 millimeters or greater than 0.8 millimeters.

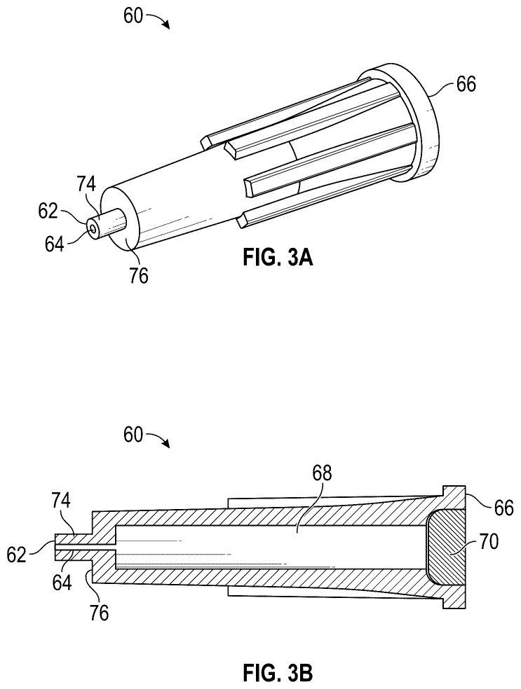

Referring now to FIGS. 3A-3B, the vent plug 60 is illustrated, according to some embodiments. In some embodiments, the distal end 62 of the vent plug 60 may include a protrusion 74 forming a stepped surface 76. In some embodiments, the hole 64 may extend through the protrusion 74. In some embodiments, the hole 64 may have a longer length than in FIGS. 2A-2C due to the protrusion 74. In some embodiments, a smaller diameter and/or length of the hole 64 may provide flow restriction and may increase a length of time it takes for blood to travel through the vent plug 60 and leak from the air-permeable plug 70. In some embodiments, the hole 64 may include a bore hole, which may be cylindrical. In some embodiments, the lumen 68 may be cylindrical or generally cylindrical.

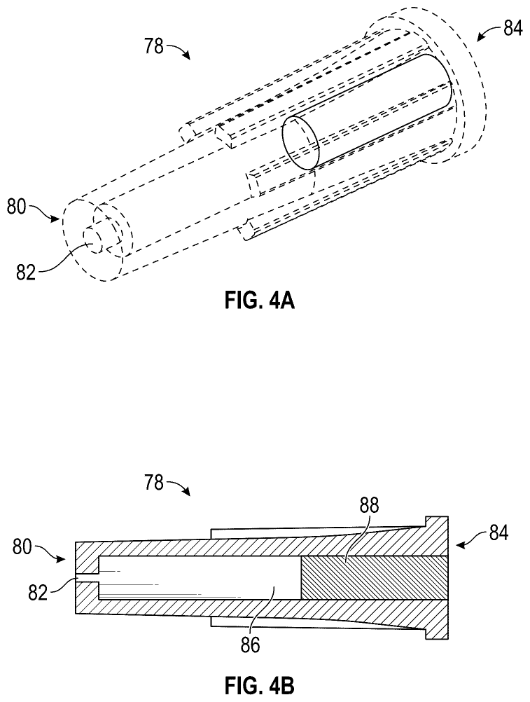

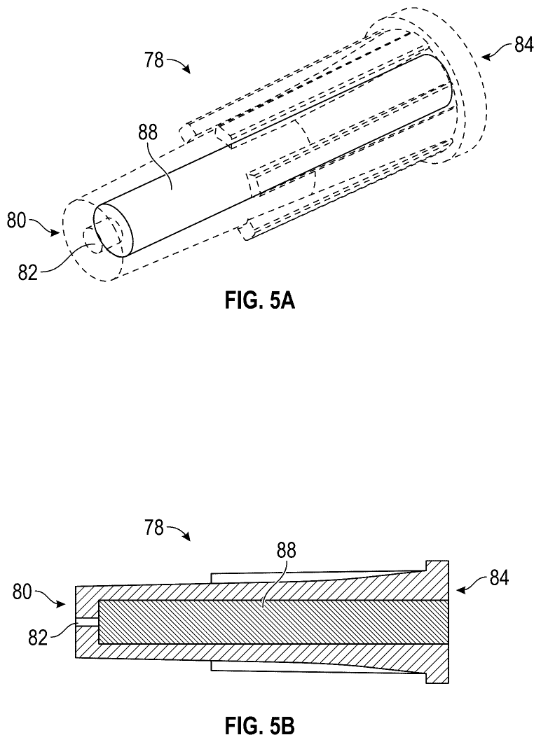

Referring now to FIGS. 4A-5B, a vent plug 78 is illustrated, according to some embodiments. In some embodiments, the vent plug 78 may be similar to the vent plug 60 of FIG. 2 and/or FIG. 3 in terms of one or more features and/or operation. In some embodiments, the vent plug 78 may include a distal end 80 having a hole 82. In some embodiments, the hole 82 may be sized to pass air but not blood. In some embodiments, the vent plug 78 may include a proximal end 84. In some embodiments, the vent plug 78 may include a lumen 86 extending proximally from the hole 82 through the proximal end 84. In some embodiments, the vent plug 78 may include an air-permeable plug 88 disposed within the lumen 86. In some embodiments, the air-permeable plug 88 may include polyurethane foam, synthetic fiber, or another suitable material. In some embodiments, the polyurethane foam may have better absorption than cellulose or filter paper and thus a time it takes for blood to travel through the vent plug 78 and leak from the air-permeable plug 70 may be increased.

In some embodiments, the diameter of the hole 82 may be less than an inner lumen of the introducer needle, which may increase the venting time and in turn provide the clinician more time to perform next steps such as capping the vent plug 78. In some embodiments, the diameter of the hole 82 may be between 0.5 millimeters and 0.8 millimeters, inclusive, which may facilitate the increase in the venting time. In some embodiments, the diameter of the hole 82 may be less than 0.5 millimeters or greater than 0.8 millimeters.

In some embodiments, as illustrated, for example, in FIGS. 4A-4B, the air-permeable plug 88 may extend along a portion of the lumen 86 and may be spaced apart from the hole 82. In some embodiments, as illustrated, for example, in FIGS. 5A-5B, the air-permeable plug 88 may extend along an entire length of the lumen 86 from the hole 82 to the proximal end 84. In these embodiments, the time it takes for blood to travel through the vent plug 78 and leak from the air-permeable plug 70 may be increased. In some embodiments, a diameter of the hole 82 may be less than a diameter of the lumen 86. In some embodiments, the hole 82 may include a bore hole, which may be cylindrical. In some embodiments, the lumen 86 may be cylindrical or generally cylindrical.

It is understood that embodiments of FIGS. 1-6 may be combined. For example, the embodiment of FIG. 4 or 5 may include the protrusion 74 of FIG. 3. All examples and conditional language recited the present disclosure are intended for pedagogical objects to aid the reader in understanding the present disclosure and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the present disclosure.

What is claimed:

1. A vent plug for a catheter system, comprising:
a distal end comprising no more than one hole, wherein the hole is sized to pass air but not blood;
a proximal end; and
a lumen extending proximally from the hole through the proximal end; and
an air-permeable plug disposed within a proximal end of the lumen, wherein the air-permeable plug comprises cellulose.

2. The vent plug of claim 1, wherein a diameter of the hole is less than a diameter of the lumen.

3. The vent plug of claim 1, wherein the distal end further comprises a protrusion forming a stepped surface, wherein the hole extends through the protrusion.

4. The vent plug of claim 1, wherein the hole comprises a cylindrical bore hole.

5. The vent plug of claim 1, wherein the lumen is cylindrical or generally cylindrical.

6. A vent plug for a catheter system, comprising:
a distal end comprising a hole, wherein the hole is sized to pass air but not blood;
a proximal end;
a lumen extending proximally from the hole through the proximal end; and
an air-permeable plug disposed within the lumen, wherein the air-permeable plug comprises polyurethane foam.

7. The vent plug of claim 6, wherein the air-permeable plug extends along a portion of the lumen and is spaced apart from the hole.

8. The vent plug of claim 6, wherein the air-permeable plug extends along an entire length of the lumen from the hole to the proximal end.

9. The vent plug of claim 6, wherein a diameter of the hole is less than a diameter of the lumen.

10. The vent plug of claim 6, wherein the hole comprises a cylindrical bore hole.

11. The vent plug of claim 6, wherein the lumen is cylindrical or generally cylindrical.

12. A catheter system, comprising:
a catheter adapter, comprising a distal end and a proximal end;
a catheter extending distally from the distal end of the catheter adapter;
a vent plug in fluid communication with the catheter, wherein the vent plug comprises:
a distal end comprising a hole, wherein the hole is sized to pass air but not blood;
a proximal end;
a lumen extending proximally from the hole through the proximal end; and
an air-permeable plug disposed within a proximal end of the lumen, wherein the air-permeable plug comprises cellulose or polyurethane foam.

13. The catheter system of claim 12, further comprising an extension tube and a connector at a proximal end of the extension tube, wherein the vent plug is coupled to the connector.

14. The catheter system of claim 13, wherein the catheter adapter further comprises a side port disposed between the distal end of the catheter adapter and the proximal end of the catheter adapter, wherein a distal end of the extension tube is coupled to the side port and the extension tube extends from the side port.

15. The catheter system of claim 13, wherein a distal end of the extension tube is coupled to the proximal end of the catheter adapter and the extension tube extends from the proximal end of the catheter adapter.

16. The catheter system of claim 12, further comprising a needle assembly coupled to the proximal end of the catheter adapter, wherein the vent plug is coupled to a proximal end of the needle assembly.

17. The catheter system of claim 12, wherein the hole comprises a cylindrical bore hole, wherein a diameter of the hole is less than a diameter of the lumen.

18. The catheter system of claim 12, wherein the distal end further comprises a protrusion forming a stepped surface, wherein the hole extends through the protrusion.

19. The catheter system of claim 12, wherein the air-permeable plug extends along a portion of the lumen and is spaced apart from the hole.

20. The catheter system of claim 12, wherein the air-permeable plug extends along an entire length of the lumen from the hole to the proximal end.
